(19) United States
(12) Patent Application Publication
(10) Pub. No.: US 2019/0219550 A1
(43) Pub. Date: Jul. 18, 2019

(54) System and Method for Identifying Shenqi Fuzheng Injection

(71) Applicant: LI MIN PHARMACEUTICAL FACTORY OF LIVZON PHARMACEUTICAL GROUP, SHAOGUAN (CN)

(72) Inventors: Yangang Song, SHAOGUAN (CN); Xuehua Liu, SHAOGUAN (CN); Wenhua Huang, SHAOGUAN (CN); Donglai Liu, SHAOGUAN (CN)

(21) Appl. No.: 16/361,136

(22) Filed: Mar. 21, 2019

Related U.S. Application Data
(63) Continuation-in-part of application No. 14/439,635, filed on Jul. 13, 2015, now abandoned.

(30) Foreign Application Priority Data
Nov. 2, 2012 (CN) 201210436493.4

(51) Int. Cl. G01N 30/86; G01N 33/15; G01N 30/72

(52) U.S. Cl. CPC G01N 30/8686; G01N 2030/8813; G01N 30/7233; G01N 33/15

(57) ABSTRACT

A system for identifying Shenqi Fuzheng injection includes a mechanism for establishing a profile of a sample to be tested; a mechanism for establishing a characteristic fingerprint profile of Shenqi Fuzheng injection as a standard fingerprint profile; and a mechanism for comparing the profile of the sample to be tested with the standard fingerprint profile to distinguish between authentic Shenqi Fuzheng injection and counterfeit Shenqi Fuzheng injection.

A method for establishing a Shenqi Fuzheng injection fingerprint spectrum, including: employing an ultra-high voltage liquid chromatography mass spectrometer to test the Shenqi Fuzheng injection, the chromatography conditions including: chromatographic column: Agilent Zorbax Eclipse Plus C18, 2.1 mm×100 mm, 1.8 μm; mobile phase: mobile phase A is 0.1% formic acid aqueous solution, and mobile phase B is 0.1% formic acid acetonitrile solution; and employing gradient elution procedure.

### CROSS-REFERENCE TO RELATED APPLICATION(S)

The present application is a continuation-in-part application of U.S. application Ser. No. 14/439,635, filed on Apr. 29, 2015, which is a National Phase Patent Application and claims priority to and the benefit of International Application Number PCT/CN2013/086405, filed on Nov. 1, 2013, which claims priority to and the benefit of Chinese Patent Application Number 201210436493.4, filed on Nov. 2, 2012, the entire contents of all of which are incorporated herein by reference.

### FIELD OF THE INVENTION

The present invention belongs to the field of drug testing, particularly relates to system and method for identifying a Shenqi Fuzheng injection.

### BACKGROUND OF THE INVENTION

Quality control of traditional Chinese herbal compound is one of the critical problems restricting development in modernization of traditional Chinese medicine. The basic theory of traditional Chinese medicine emphasizes the overall effect of the medicines and attaches importance to the synergistic effects on efficacy. Taking one or two effective components in Chinese medicines as qualitative and quantitative indexes is far from effectively controlling and assessing the quality of Chinese medicines, more difficult to reflect the safety and effectiveness of Chinese medicines. Traditional Chinese herbal compound is a compound preparation, which combines more than two kinds of Chinese medicinal herbs to be used in disease treatment, whose quality control is more difficult than single Chinese medicinal herb. In recent years, Chinese medicine fingerprint profile and characteristic fingerprint profile are widely used in quality control, wherein the Chinese medicine characteristic fingerprint profile refers to a characteristic fingerprint profile made up by selecting several chromatographic peaks with good specificity or a combination of chromatographic peaks with good specificity from the Chinese medicine fingerprint profile, which can be used to monitor the quality of Chinese medicines by observing the presence or absence and changes of the characteristic fingerprint peaks. In Chinese Pharmacopoeia, the 2010 Edition, the characteristic fingerprint profile has been widely applied in quality control of intermediate components of various Chinese medicines.

Ultra high performance liquid chromatography (UHPLC) technology is a great breakthrough of the chromatography technique. It has advantages in ultra-high speed, ultra-high sensitivity and ultra-high resolution, and it achieves faster and more sensitive detection performance by using a smaller chromatographic column packing technology. It is widely used in pesticide residues and drug metabolism in foreign countries, and its application become more and more common in China.

Chromatography-mass spectrometry technology is an advanced analysis technique developing rapidly in recent years, wherein, after extensive application of the gas chromatography-mass spectrometry (GC-MS) technology, the liquid chromatography-mass spectrometry (LC-MS) technology is an another technique which is gradually recognized and accepted, but it is not yet widely applied because of expensive equipment. The ionization technology adopted in liquid chromatography-mass spectrometers can not only resolve the problems in detecting some ingredients without ultraviolet absorption, such as saponins, but also get a precise molecular weight of the ionized component, which provides data support for identification and confirmation of the component and the structure thereof.

Shenqi Fuzheng injection is a Chinese medicine infusion, with effect of benefiting qi for strengthening resistance, and is used for the treatment of lassitude, shortness of breath with no desire to speak, spontaneous sweating and vertigo caused by lung-spleen deficiency, and used as an adjuvant therapy for lung cancer and gastric cancer patients with the syndromes above. It is one of National Protection Varieties of Traditional Chinese Medicine, and the Protection Variety No. is: ZYB2072004073. Among quality standards for Shenqi Fuzheng injection, the “Fingerprint Profile” item saying detection by using high performance liquid chromatography-ultraviolet detector has some limitations, for example, the main component saponin has no ultraviolet absorption. The component saponin may be monitored by using high performance liquid chromatography-evaporative light scattering detector, but pre-treatments for samples are complicated and furthermore the analysis takes a long time. Therefore, the main components cannot be monitored comprehensively and quickly, and there needs to be improved.

### SUMMARY OF THE INVENTION

The technical problem to be solved in the present invention is to remedy deficiencies in prior art, and the objective of the present invention is to provide a method for establishing Shenqi Fuzheng injection fingerprint profile. The fingerprint profile established by the method described in the present invention can be used as a standard fingerprint profile to be applied in identification of Shenqi Fuzheng injection.

The present invention achieves the above objective by use of the following technical solutions:

A method for establishing Shenqi FuZheng injection fingerprint profile, wherein the method comprises testing Shenqi Fuzheng injection by ultra-high pressure liquid chromatography-mass spectrometer, for example ultra-high performance liquid chromatography-quadrupole time-of-flight mass spectrometer, wherein the chromatographic conditions include the followings:

Chromatographic column: Agilent Zorbax Eclipse Plus C18, 2.1 mm×100 mm, 1.8 μm;

Mobile phase: Mobile phase A is 0.1% (v/v) formic acid aqueous solution, mobile phase B is 0.1% (v/v) formic acid acetonitrile solution;

Using gradient elution according to the following elution program, wherein the proportions of the mobile phases are all volume percentages:

0-5 min, mobile phase A is 95%, mobile phase B is 5%;

0.5-10 min, mobile phase A is 95%-75%, mobile phase B is 5%-25%;

10-15 min, mobile phase A is 75%-45%, mobile phase B is 25%-55%;

15-18 min, mobile phase A is 45%-0%, mobile phase B is 55%-100%;

18-20 min, mobile phase A is 0%, mobile phase B is 100%.

Preferably, the chromatographic conditions further include the followings:

Flow rate: 0.35 ml/min;

Column temperature: 40° C.;

Injection volume: 5 μl.

Preferably, in the method described above for establishing Shenqi FuZheng injection fingerprint profile, the mass spectrometry conditions include the followings:

The ion source is an ESI source, and detection is operated in negative ion mode;

Atomized gas pressure: 35 psig;

Dry gas temperature: 350° C.;

Dry gas flow rate: 10 L/min;

Capillary voltage: 3,500 V;

Voltage at capillary exit: 135 V.

Preferably, the method described above for establishing Shenqi Fuzheng injection fingerprint profile further comprises preparation of control solutions by following steps:

Accurately weighing an appropriate amount of calycosin glucoside or astragaloside IV, and then adding methanol to prepare a solution containing 0.004 mg of calycosin glucoside per ml or 0.006 mg of astragaloside IV per ml, respectively.

Preferably, the method described above for establishing Shenqi FuZheng injection fingerprint profile further comprises the preparation of test sample solution by the following step: filtering the Shenqi Fuzheng injection through a 0.22 μm microporous filter membrane.

Preferably, the method described above for establishing Shenqi FuZheng injection fingerprint profile comprises the following steps:

(1) Preparation of control solution: Accurately weighing an appropriate amount of calycosin glucoside or astragaloside IV, then adding methanol to prepare a solution containing 0.004 mg of calycosin glucoside per ml or 0.006 mg of astragaloside IV per ml, respectively;

(2) Preparation of test sample solution: Filtering Shenqi Fuzheng injection through a 0.22 μm microporous filter membrane;

(3) Determination: Accurately aspirating 5 μl of the control solution or the test sample solution, respectively, and then injecting the solutions into a ultra-high pressure liquid chromatography-mass spectrometer, for example ultra-high performance liquid chromatography-quadrupole time-of-flight mass spectrometer, conducting determination according to the following conditions to obtain the Shenqi Fuzheng injection fingerprint profile:

wherein the chromatographic conditions include the followings:

Chromatographic column: Agilent Zorbax Eclipse Plus C18, 2.1 mm×100 mm, 1.8 μm;

Mobile phase: mobile phase A is 0.1% (v/v) formic acid aqueous solution, mobile phase B is 0.1% (v/v) formic acid acetonitrile solution;

Using gradient elution according to the following elution program, wherein the proportions of the mobile phases are all volume percentages:

0-5 min, mobile phase A is 95%, mobile phase B is 5%;

0.5-10 min, mobile phase A is 95%-75%, mobile phase B is 5%-25%;
10-15 min, mobile phase A is 75%-45%, mobile phase B is 25%-55%;
15-18 min, mobile phase A is 45%-0%, mobile phase B is 55%-100%;
18-20 min, mobile phase A is 0%, mobile phase B is 100%;
Preferably, the chromatographic conditions further include the followings:
Flow rate: 0.35 ml/min;

Column temperature: 40° C.;

Preferably, the mass spectrometry conditions include the followings:

The ion source is an ESI source, and detection is operated in negative ion mode;

Atomized gas pressure: 35 psig;

Dry gas temperature: 350° C.;

Dry gas flow rate: 10 L/min;

Capillary voltage: 3,500 V;

Voltage at capillary exit: 135 V.

Preferably, the method described above for establishing Shenqi Fuzheng

injection fingerprint profile further comprises:

Comparing multiple Shenqi Fuzheng injection fingerprint profiles, picking out common characteristic peaks to obtain the Shenqi Fuzheng injection characteristic fingerprint profile.

Preferably, in the method described above for establishing Shenqi Fuzheng injection fingerprint profile, the Shenqi Fuzheng injection fingerprint profile or Shenqi Fuzheng injection characteristic fingerprint profile comprises 18 characteristic peaks, the retention time of each characteristic peak is as follows:

Peak 1: 7.1 min, Peak 2: 7.5 min, Peak 3: 8.1 min, Peak 4: 8.6 min, Peak 5: 9.2 min, Peak 6: 9.9 min, Peak 7: 10.9 min, Peak 8: 11.3 min, Peak 9: 11.7 min, Peak 10: 12.7 min, Peak 11: 13.4 min, Peak 12: 13.7 min, Peak 13: 14.4 min, Peak 14: 14.8 min, Peak 15: 15.1 min, Peak 16: 15.5 min, Peak 17: 15.9 min, Peak 18: 16.3 min.

Preferably, in the method described above for establishing Shenqi Fuzheng injection fingerprint profile, the Shenqi Fuzheng injection fingerprint profile or the Shenqi Fuzheng injection characteristic fingerprint profile takes the control astragaloside IV as a reference peak, by which the relative retention time of each characteristic peak is calculated, as follows:

Peak 1: 0.52, Peak 2: 0.54, Peak 3: 0.59, Peak 4: 0.62, Peak 5: 0.66, Peak 6: 0.72, Peak 7: 0.79, Peak 8: 0.82, Peak 9: 0.85, Peak 10: 0.92, Peak 11: 0.97, Peak 12: 1.00, Peak 13: 1.04, Peak 14: 1.07, Peak 15: 1.10, Peak 16: 1.13, Peak 17: 1.16, Peak 18: 1.19.

Preferably, in the Shenqi Fuzheng injection fingerprint profile or the Shenqi Fuzheng injection characteristic fingerprint profile, Peak 2 and Peak 12 are calycosin glucoside and astragaloside IV, respectively; preferably, the ratio of the area of calycosin glucoside peak and the astragaloside IV peak to the area of the corresponding reference peak is 0.5-1.5.

The present invention also provide a method for identifying Shenqi Fuzheng injection, wherein the method comprises comparing the fingerprint profile or the characteristic fingerprint profile of the test sample established according to the method described above with the standard fingerprint profile or the characteristic fingerprint profile established according to the method described above so as to identify authenticity.

In a preferred embodiment, the present invention provides a method for establishing Shenqi Fuzheng injection fingerprint profile, wherein the method comprises the following steps:

Preparing a mixed control solution of calycosin glucoside and astragaloside IV, containing calycosin glucoside at a concentration of 0.004 mg/ml and astragaloside IV at a concentration of 0.006 mg/ml;

Taking a filtrate of Shenqi Fuzheng injection as a test solution; Analyzing the control solution and the test sample solution described above by ultra high performance liquid chromatography-quadrupole time-of-flight mass spectrometer, wherein the chromatographic conditions include the followings:

Chromatographic column: Agilent Zorbax Eclipse Plus C18, 2.1 mm×100 mm, 1.8 μm;

Mobile phase: mobile phase A is 0.1% (v/v) formic acid aqueous solution, mobile phase B is 0.1% (v/v) formic acid acetonitrile solution;

Gradient elution;

Flow rate: 0.35 ml/min;

Column temperature: 40° C.;

Injection volume: 5 μl;

Ultra high performance liquid chromatography-quadrupole time-of-flight mass spectrometry fingerprint profile (total ion chromatogram profile) for Shenqi Fuzheng injection is obtained;

The steps of gradient elution include the followings: 0-5 min, mobile phase acetonitrile-water is 5:95; 0.5-10 min, mobile phase acetonitrile-water changes from 5:95 to 25:75 gradually; 10-15 min, mobile phase acetonitrile-water changes from 25:75 to 55:45 gradually; 15-18 min, mobile phase acetonitrile-water changed from 55:45 to 100:0 gradually; 18-20 min, mobile phase acetonitrile-water is 100:0.

Shenqi Fuzheng injection characteristic fingerprint profile is determined according to the fingerprint profile (total ion chromatogram profile) and the characteristic fingerprint profile (extracted ion chromatogram profile) extracted from the fingerprint profile (total ion chromatogram profile), and thereby monitoring the quality of Shenqi Fuzheng injection.

The gradient elution steps can also be shown in Table 1:

TABLE 1
Gradient Elution Program

| Time (min) | Mobile Phase A (%) | Mobile Phase B (%) |
|---|---|---|
| 0-0.5 | 95 | 5 |
| 0.5-10 | 95 → 75 | 5 → 25 |
| 10-15 | 75 → 45 | 25 → 55 |
| 15-18 | 45 → 0 | 55 → 100 |
| 18-20 | 0 | 100 |

According to the method in the present invention, total ion chromatograms (TIC) of 100 batches of Shenqi Fuzheng injection are analyzed and compared to sorted out the common characteristic peaks, the ion mass numbers of which are used to obtain the extracted ion chromatograms (EIC), followed by marking the retention time (Rt) of each common characteristic peak, so as to obtain the Shenqi Fuzheng injection characteristic fingerprint profile.

There are 18 common characteristic peaks, with a retention time (Rt) and a mass number as follows: 7.1 min (471.2083), 7.5 min (491.1195), 8.1 min (441.1919), 8.6 min (309.1555), 9.2 min (187.0976), 9.9 min (441.1766), 10.9 min (593.1876), 11.3 min (507.1508), 11.7 min (463.1610), 12.7 min (991.5119), 13.4 min (991.5119), 13.7 min (829.4591), 14.4 min (871.4697), 14.8 min (871.4697), 15.1 min (871.4697), 15.5 min (913.4650), 15.9 min (913.4650), 16.3 min (913.4650), respectively, wherein the chromatographic peaks with a Rt of 7.5 min and 13.7 min are verified as calycosin glucoside and astragaloside IV, respectively; Peak S is a peak corresponding to the astragaloside IV reference peak; the relative retention time of each characteristic peak is calculated, wherein the retention time should fluctuate within ±5% of the specified values, which are listed in an order of 0.52, 0.54, 0.59, 0.62, 0.66, 0.72, 0.79, 0.82, 0.85, 0.92, 0.97, 1.00, 1.04, 1.07, 1.10, 1.13, 1.16, 1.19; wherein the ratio of the peak area of calycosin glucoside and astragaloside IV to the peak area of their corresponding reference should be within 0.5-1.5.

The characteristic fingerprint profile established by the method of the present invention can be used in identifying Shenqi Fuzheng injection.

The present invention also provides a method for detecting saponins in Shenqi Fuzheng injection by HPLC-ELSD method, wherein the chromatographic conditions include the followings:

Chromatographic column: Discovery C-18, 5 μm, 250×4.6 mm;

Mobile phase: Mobile phase A is acetonitrile, mobile phase B is water;

Using gradient elution according to the following elution program, wherein the proportions of the mobile phases are all volume percentages:

0-30 min, mobile phase A is 12-25%, mobile phase B is 88-75%;

30-40 min, mobile phase A is 25%-32%, mobile phase B is 75%-68%;

40-55 min, mobile phase A is 32%-55%, mobile phase B is 68%-45%;

55-68 min, mobile phase A is 55%-90%, mobile phase B is 45%-10%;

68-72 min, mobile phase A is 90%, mobile phase B is 10%;

Preferably, wherein, the chromatographic conditions also include the followings:

Flow rate: 1.0 ml/min;

Column temperature: 30° C.;

Injection volume: 20 μl.

Preferably, wherein the conditions of the ELSD detector include the followings: the temperature of drift tube was 55° C.;

atomizing power was 60%;

gain value was 20;

gas pressure was 25 psig.

Preferably, wherein said method further comprises preparation of control solutions by the following steps:

accurately weighing an appropriate amount of astragaloside IV, and adding methanol to prepare a solution containing 0.10 mg of astragaloside IV per ml.

Preferably, wherein the method further comprises preparation of a test sample solution by the following step:

Shenqi Fuzheng Injection was fractionated on a macroporous resin, eluted with water and firstly 30 v/v % ethanol solution and then 70 v/v % ethanol solution to remove polysaccharides, protein and salts, and then concentrated, made up to the mark, filtered.

According to an embodiment, a system for identifying Shenqi Fuzheng injection includes a mechanism for establishing a profile of a sample to be tested; a mechanism for establishing a characteristic fingerprint profile of Shenqi Fuzheng injection as a standard fingerprint profile; and a mechanism for comparing the profile of the sample to be tested with the standard fingerprint profile to distinguish between authentic Shenqi Fuzheng injection and counterfeit Shenqi Fuzheng injection. The standard fingerprint profile includes 18 characteristic peaks, and a retention time and a mass number of the 18 characteristic peaks are as follows:

Peak 1:7.1 min (471.2083), Peak 2: 7.5 min (491.1195), Peak 3: 8.1 min (441.1919), Peak 4: 8.6 min (309.1555), Peak 5: 9.2 min (187.0976), Peak 6: 9.9 min (441.1766), Peak 7: 10.9 min (593.1876), Peak 8: 11.3 min (507.1508), Peak 9: 11.7 min (463.1610), Peak 10: 12.7 min (991.5119), Peak 11: 13.4 min (991.5119), Peak 12: 13.7 min (829.4591), Peak 13: 14.4 min (871.4697), Peak 14: 14.8 min (871.4697), Peak 15: 15.1 min (871.4697), Peak 16: 15.5 min (913.4650), Peak 17: 15.9 min (913.4650), and Peak 18: 16.3 min (913.4650);

Peak 2 and Peak 12 are calycosin glucoside and astragaloside IV, respectively. The Shenqi Fuzheng injection characteristic fingerprint profile takes a control astragaloside IV as a reference peak, by which a relative retention time of each characteristic peak is calculated, as follows:

Peak 1: 0.52, Peak 2: 0.54, Peak 3: 0.59, Peak 4: 0.62, Peak 5: 0.66, Peak 6: 0.72, Peak 7: 0.79, Peak 8: 0.82, Peak 9: 0.85, Peak 10: 0.92, Peak 11: 0.97, Peak 12: 1.00, Peak 13: 1.04, Peak 14: 1.07, Peak 15: 1.10, Peak 16: 1.13, Peak 17: 1.16, and Peak 18: 1.19.

When comparing, the sample is identified as authentic Shenqi Fuzheng injection if the relative retention time fluctuates within ±5% of the above specified values, and a ratio of an area of calycosin glucoside peak and the astragaloside IV peak to an area of a corresponding reference peak is 0.5-1.5.

The mechanism for establishing the profile of the samples to be tested and the mechanism for establishing the characteristic fingerprint profile of Shenqi Fuzheng injection as the standard fingerprint profile may each independently include ultra-high pressure liquid chromatography-mass spectrometer.

The system may further include a mechanism for detecting saponins in Shenqi Fuzheng injection.

Compared with the prior art, the present invention has beneficial effects as follows:

In the quality standards for Shenqi Fuzheng injection, the “Fingerprint Profile” item says that Shenqi Fuzheng injection fingerprint profile is determined by high performance liquid chromatography-UV detection, which achieves the objective of monitoring the quality to a certain extent, but the main component saponins substantially has no absorption under ultraviolet; the “Content Determination” item says that the content of total saponins is determined by UV spectrophotometry with vanillin-glacial acetic acid, and the content of astragaloside IV is determined by high performance liquid chromatography-evaporative light scattering detection, but these still cannot fully reflect the content of each saponin component. In the internal control in enterprise, the saponin component can be monitored by further using high performance liquid chromatography-evaporative light scattering detector to determine the fingerprint profile, but pre-treatments for samples are required additionally, and the analysis would take a long time.

Therefore, the technical standards for Shenqi Fuzheng injection characteristic fingerprint profile established by ultra high performance liquid chromatography-quadrupole time-of-flight mass spectrometry according to the method of the present invention can make it easier to monitor drug quality comprehensively, quickly, and effectively, depending on presence or absence and characteristics of common peaks in the characteristic fingerprint profile, so as to ensure stable, uniform and controllable qualities. The present invention has features in is advanced method and better stability and reproducibility.

### BRIEF DESCRIPTION OF THE DRAWINGS

FIG. 1 illustrates a total ion chromatogram (TIC) of the Shenqi Fuzheng injection provided by the present invention, wherein the arrows from left to right indicate the characteristic peaks 1 to 18, respectively;

FIG. 2 illustrates an extracted ion chromatogram (EIC) of the Shenqi Fuzheng injection, wherein the arrows from left to right indicate the characteristic peaks 1 to 18, respectively;

FIG. 3 illustrates an extracted ion chromatogram (EIC) of the control mixture, wherein Peaks 2 and 12 represent calycosin glucoside and astragaloside IV, sequentially;

FIG. 4 is a graph illustrating the comparison of the Shenqi Fuzheng injection fingerprint profile of the present invention with a counterfeit, wherein “1” showing the certified Shenqi Fuzheng injection, “2” showing the counterfeit (presumed as Danshen Injection), and “3” showing a Danshen infusion solution;

FIG. 5 is a chromatogram obtained by using the mobile phase shown in table 8;

FIG. 6 is a chromatogram obtained by using the mobile phase shown in table 9;

FIG. 7 is a chromatogram obtained by using the mobile phase shown in table 10;

FIG. 8 is a chromatogram obtained by using the mobile phase shown in table 11;

FIG. 9 is a chromatogram obtained on Kromasil 100-5 C18 column;

FIG. 10 is a chromatogram obtained on Diamonsil C18 column;

FIG. 11 is a chromatogram obtained on Discovery C18 column;

FIG. 12 is a chromatogram obtained at a flow rate of 0.8 ml/min;

FIG. 13 is a chromatogram obtained at a flow rate of 1.0 ml/min;

FIG. 14 is a chromatogram obtained at a flow rate of 1.2 ml/min;

FIG. 15 is a chromatogram with an injection volume of 10 μl;

FIG. 16 is a chromatogram with an injection volume of 20 μl;

FIG. 17 is a chromatogram obtained by extracting with n-butanol;

FIG. 18 is a chromatogram obtained by purifying on D₁₀₁ macroporous resin directly;

FIG. 19 is a chromatogram obtained by purifying on D₁₀₁ macroporous resin after concentration;

FIG. 20 is a chromatogram obtained after 120 min;

FIG. 21 is a chromatogram of astragaloside IV;

FIG. 22 is a chromatogram of calycosin glucoside;

FIG. 23 is a chromatogram of negative control solution without radix astragali;

FIG. 24 is a chromatogram of negative control solution without radix codonopsis;

FIG. 25 is a chromatogram of negative control solution without radix astragali and radix codonopsis;

FIG. 26 is control fingerprint of saponins of Shenqi Fuzheng Injection (Peak S: astragaloside IV);

FIG. 27 is a spectrum of Shenqi Fuzheng Injection by HPLC-ELSD method;

FIG. 28 is a total ionic spectrum of Shenqi Fuzheng Injection by HPLC-MS, wherein Peak 1: Astragaloside V, Peak 2: Astragaloside VI, Peak 3: Astragaloside IV, Peak 4: Isoastragaloside II, Peak 5: Astragaloside II, Peak 6: Cyclocephaloside II,

FIG. 29 is a spectrum of control solution of Astragaloside V by HPLC-ELSD method;

FIG. 30 is a spectrum of control solution of Astragaloside VI by HPLC-ELSD method;

FIG. 31 is a spectrum of control solution of Astragaloside IV by HPLC-ELSD method;

FIG. 32 is a spectrum of control solution of Isoastragaloside II by HPLC-ELSD method;

FIG. 33 is a spectrum of control solution of Astragaloside II by HPLC-ELSD method; and

FIG. 34 is a spectrum of control solution of Cyclocephaloside II by HPLC-ELSD method.

### DETAILED DESCRIPTION OF THE EMBODIMENTS

The technical solutions of the present invention will be further described in details in combination with specific examples.

### Example 1

1. Instrument and Test Drug

1.1 Instruments:

Agilent LC/MSD (1290UHPLC dual-gradient pump, built-in vacuum degasser, 100-bit automatic sampler, intelligent column oven, high-precision quadrupole tandem time-of-flight mass spectrometer system); Chromatographic column: Agilent Zorbax Eclipse Plus C18 (2.1 mm×100 mm, 1.8 μm).

1.2 Test Drug:

Shenqi Fuzheng injection, provided by Livzon Group Limin Pharmaceutical Factory. Reagents acetonitrile and formic acid used in the experiments were both chromatographically pure, and the water was ultrapure water.

2. Method and Result

2.1 Preparation of Test Sample Solution:

Shenqi Fuzheng injection was filtered through a 0.22 μm microporous filter membrane.

2.2 Preparation of Mixed Control Solution:

An appropriate amount of calycosin glucoside and astragaloside IV was accurately weighed, and methanol was then added to prepare a solution containing 0.004 mg of calycosin glucoside and 0.006 mg of astragaloside IV per ml, respectively.

2.3 Chromatographic Conditions:

Chromatographic column was Agilent Zorbax Eclipse Plus C18, 2.1 mm×100 mm, 1.8 μm; Mobile phases were 0.1% formic acid aqueous solution (A) and 0.1% formic acid acetonitrile solution (B); Column temperature was 40° C.; Injection volume was 5 μl; A gradient elution program as shown in Table 2 was used:

TABLE 2
Gradient Elution Program

| Time (min) | Mobile Phase A (%) | Mobile Phase B (%) |
|---|---|---|
| 0-0.5 | 95 | 5 |
| 0.5-10 | 95 → 75 | 5 → 25 |
| 10-15 | 75 → 45 | 25 → 55 |
| 15-18 | 45 → 0 | 55 → 100 |
| 18-20 | 0 | 100 |

2.4 Mass Spectrometry Conditions:

The ion source was an ESI source, detection was operated in negative ion mode; Atomized gas pressure: 35 psig; Dry gas temperature: 350° C.; Dry gas flow rate: 10 L/min; Vcap Capillary voltage: 3,500 V; Voltage at capillary exit: 135 V.

2.5 Determination Method

5 μl of the control solution or the test sample solution was accurately aspirated, respectively, then injected into the liquid chromatography-mass spectrometer, respectively, determination was conducted, recording the spectra for 20 minutes.

2.6 Determination of Common Characteristic Peaks

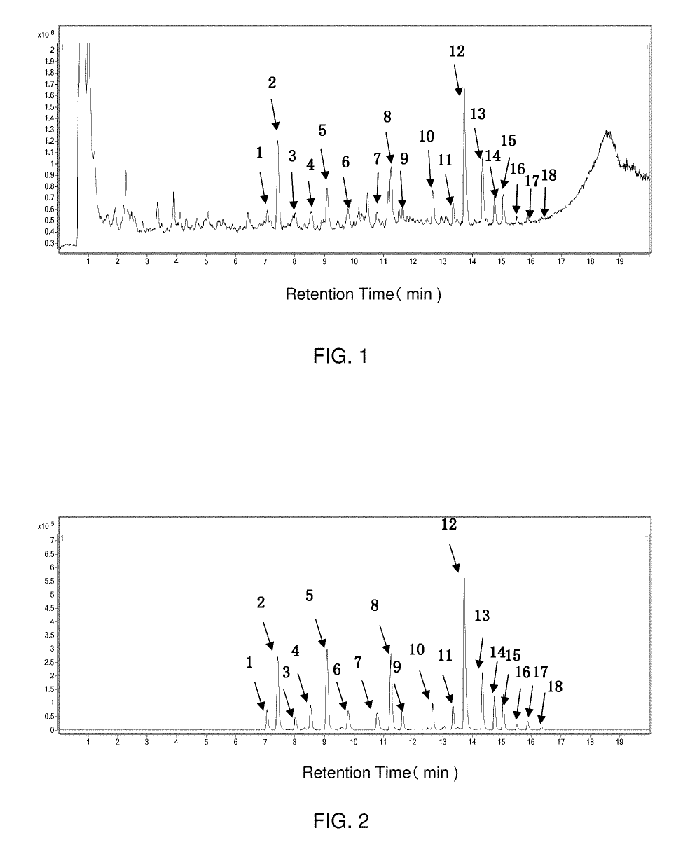

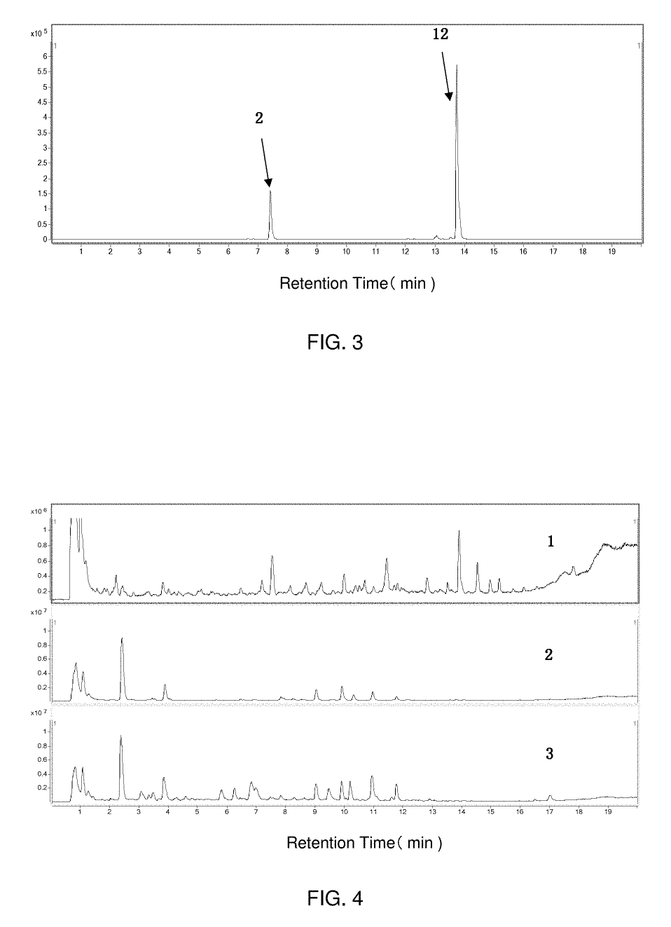

The common characteristic peaks were sorted out by comparing the total ion chromatograms (TIC) of 100 batches of Shenqi Fuzheng injection, see details in FIG. 1; the extracted ion chromatogram (EIC) was obtained by using the ion mass number of these common characteristic peaks, see details in FIG. 2 (specifically, a series of target ions were extracted from FIG. 1 by using the qualitative analysis software in the data analysis software Masshunter and utilizing the function of ion extraction, and thereby obtaining FIG. 2); then the retention time (Rt) of each common characteristic peak was marked, so as to obtain the Shenqi Fuzheng injection characteristic fingerprint profile. There were 18 common characteristic peaks, with a retention time (Rt) and a mess number as follows: 7.1 min (471.2083), 7.5 min (491.1195), 8.1 min (441.1919), 8.6 min (309.1555), 9.2 min (187.0976), 9.9 min (441.1766), 10.9 min (593.1876), 11.3 min (507.1508), 11.7 min (463.1610), 12.7 min (991.5119), 13.4 min (991.5119), 13.7 min (829.4591), 14.4 min (871.4697), 14.8 min (871.4697), 15.1 min (871.4697), 15.5 min (913.4650), 15.9 min (913.4650), 16.3 min (913.4650), wherein the chromatographic peaks with a Rt of 7.5 min and 13.7 min were verified as calycosin glucoside and astragaloside IV respectively, see details in FIG. 3; Peak S was a peak corresponding to an astragaloside IV reference peak, the retention time of each characteristic peaks was calculated, wherein the retention time should fluctuate within ±5% of the specified values, which were listed in an order of 0.52, 0.54, 0.59, 0.62, 0.66, 0.72, 0.79, 0.82, 0.85, 0.92, 0.97, 1.00, 1.04, 1.07, 1.10, 1.13, 1.16, 1.19; the ratio of the peak area of calycosin glucoside and astragaloside IV to the peak area of their corresponding reference should be within 0.5-1.5.

2.7 Precision Assay:

The test sample solution from the same Shenqi Fuzheng injection was injected for continuously 6 times, followed by extracting the characteristic peaks and marking the retention time. Peak 12 of astragaloside IV was taken as Peak S, and then the relative retention time of each of other characteristic peaks were calculated. The results showed that the RSD values of the relative retention time of each characteristic peak was all less than 1%, and the precision of the instrument was excellent. The results of the precision assay are shown in Table 3:

TABLE 3
Results of Precision Assay

| | 1 | 2 | 3 | 4 | 5 | 6 | RSD (%) |
|---|---|---|---|---|---|---|---|
| 1 | 0.517 | 0.517 | 0.517 | 0.516 | 0.516 | 0.517 | 0.14 |
| 2 | 0.542 | 0.542 | 0.542 | 0.541 | 0.541 | 0.542 | 0.09 |
| 3 | 0.586 | 0.586 | 0.586 | 0.585 | 0.585 | 0.586 | 0.08 |
| 4 | 0.623 | 0.624 | 0.624 | 0.623 | 0.623 | 0.624 | 0.08 |
| 5 | 0.661 | 0.661 | 0.661 | 0.661 | 0.661 | 0.661 | 0.04 |
| 6 | 0.716 | 0.715 | 0.716 | 0.715 | 0.716 | 0.716 | 0.06 |
| 7 | 0.786 | 0.786 | 0.787 | 0.787 | 0.788 | 0.787 | 0.08 |
| 8 | 0.820 | 0.819 | 0.820 | 0.820 | 0.820 | 0.821 | 0.08 |
| 9 | 0.847 | 0.847 | 0.848 | 0.848 | 0.849 | 0.848 | 0.06 |
| 10 | 0.923 | 0.922 | 0.923 | 0.922 | 0.923 | 0.923 | 0.01 |
| 11 | 0.972 | 0.972 | 0.972 | 0.972 | 0.972 | 0.972 | 0.02 |
| S | 1.000 | 1.000 | 1.000 | 1.000 | 1.000 | 1.000 | 0.00 |
| 13 | 1.044 | 1.044 | 1.044 | 1.045 | 1.044 | 1.044 | 0.02 |
| 14 | 1.074 | 1.074 | 1.074 | 1.074 | 1.074 | 1.074 | 0.02 |
| 15 | 1.096 | 1.096 | 1.096 | 1.096 | 1.096 | 1.096 | 0.01 |
| 16 | 1.129 | 1.129 | 1.128 | 1.129 | 1.129 | 1.129 | 0.03 |
| 17 | 1.155 | 1.155 | 1.155 | 1.156 | 1.156 | 1.155 | 0.02 |
| 18 | 1.189 | 1.189 | 1.189 | 1.189 | 1.189 | 1.189 | 0.02 |

2.8 Stability Assay:

The test sample solution from the same Shenqi Fuzheng injection was injected at 0 hour, at 1 hour, at 2 hours, at 4 hours, at 8 hours, and at 12 hours, respectively, followed by extracting the characteristic peaks and marking the retention time. Peak 12 of astragaloside IV was taken as Peak S, and then the relative retention time of each of other characteristic peaks was calculated. The results showed that the RSD values of the relative retention time of each characteristic peak was all less than 1%), and the test sample solution remained stable within 12 hours during standing. The results of the stability assay are shown in Table 4:

TABLE 4
Results of Stability Assay

| | 0 | 1 | 2 | 4 | 8 | 12 | RSD (%) |
|---|---|---|---|---|---|---|---|
| 1 | 0.517 | 0.516 | 0.516 | 0.517 | 0.517 | 0.517 | 0.15 |
| 2 | 0.542 | 0.541 | 0.541 | 0.542 | 0.541 | 0.543 | 0.11 |
| 3 | 0.586 | 0.586 | 0.585 | 0.586 | 0.586 | 0.587 | 0.09 |
| 4 | 0.624 | 0.623 | 0.623 | 0.624 | 0.623 | 0.624 | 0.09 |
| 5 | 0.662 | 0.662 | 0.661 | 0.662 | 0.661 | 0.662 | 0.05 |
| 6 | 0.716 | 0.716 | 0.715 | 0.716 | 0.715 | 0.716 | 0.06 |
| 7 | 0.787 | 0.786 | 0.786 | 0.788 | 0.787 | 0.786 | 0.07 |
| 8 | 0.820 | 0.820 | 0.819 | 0.820 | 0.820 | 0.821 | 0.04 |
| 9 | 0.848 | 0.848 | 0.847 | 0.848 | 0.848 | 0.847 | 0.04 |
| 10 | 0.922 | 0.922 | 0.923 | 0.923 | 0.922 | 0.923 | 0.02 |
| 11 | 0.972 | 0.972 | 0.972 | 0.972 | 0.972 | 0.972 | 0.01 |
| S | 1.000 | 1.000 | 1.000 | 1.000 | 1.000 | 1.000 | 0.00 |
| 13 | 1.044 | 1.044 | 1.044 | 1.044 | 1.044 | 1.045 | 0.01 |
| 14 | 1.074 | 1.074 | 1.074 | 1.074 | 1.074 | 1.074 | 0.01 |
| 15 | 1.096 | 1.096 | 1.096 | 1.096 | 1.096 | 1.096 | 0.02 |
| 16 | 1.129 | 1.129 | 1.129 | 1.129 | 1.129 | 1.129 | 0.02 |
| 17 | 1.155 | 1.156 | 1.156 | 1.155 | 1.156 | 1.156 | 0.02 |
| 18 | 1.189 | 1.190 | 1.189 | 1.189 | 1.189 | 1.190 | 0.04 |

2.9 Repeatability Assay:

Six Shenqi Fuzheng injection of the same batch were taken, prepared according to the method of preparing the test sample solution, and then injected, respectively, followed by extracting the characteristic peaks and marking the retention time. Peak 12 of astragaloside IV was taken as peak S, and then the relative retention time of each of other characteristic peaks was calculated, The results showed that the RSD values of the relative retention time of each characteristic peak was all less than 1%, and the method has good repeatability. The results of the repeatability assay are shown in Table 5:

TABLE 5
Results of Repeatability Test

| | 1 | 2 | 3 | 4 | 5 | 6 | RSD (%) |
|---|---|---|---|---|---|---|---|
| 1 | 0.512 | 0.511 | 0.511 | 0.512 | 0.511 | 0.517 | 0.45 |
| 2 | 0.537 | 0.536 | 0.536 | 0.536 | 0.536 | 0.542 | 0.45 |
| 3 | 0.581 | 0.581 | 0.580 | 0.580 | 0.581 | 0.586 | 0.40 |
| 4 | 0.619 | 0.619 | 0.618 | 0.619 | 0.618 | 0.624 | 0.38 |
| 5 | 0.655 | 0.655 | 0.655 | 0.655 | 0.655 | 0.661 | 0.37 |
| 6 | 0.711 | 0.710 | 0.710 | 0.710 | 0.709 | 0.716 | 0.36 |
| 7 | 0.782 | 0.782 | 0.781 | 0.782 | 0.781 | 0.786 | 0.25 |
| 8 | 0.815 | 0.815 | 0.815 | 0.815 | 0.815 | 0.820 | 0.25 |
| 9 | 0.843 | 0.844 | 0.844 | 0.844 | 0.844 | 0.848 | 0.18 |
| 10 | 0.922 | 0.922 | 0.922 | 0.922 | 0.922 | 0.923 | 0.03 |
| 11 | 0.972 | 0.972 | 0.972 | 0.972 | 0.972 | 0.972 | 0.01 |
| S | 1.000 | 1.000 | 1.000 | 1.000 | 1.000 | 1.000 | 0.00 |
| 13 | 1.045 | 1.044 | 1.045 | 1.044 | 1.044 | 1.044 | 0.02 |
| 14 | 1.074 | 1.075 | 1.075 | 1.075 | 1.074 | 1.074 | 0.03 |
| 15 | 1.096 | 1.097 | 1.097 | 1.096 | 1.096 | 1.096 | 0.04 |
| 16 | 1.129 | 1.129 | 1.130 | 1.129 | 1.128 | 1.129 | 0.05 |
| 17 | 1.156 | 1.156 | 1.156 | 1.156 | 1.155 | 1.155 | 0.04 |
| 18 | 1.190 | 1.190 | 1.190 | 1.190 | 1.190 | 1.189 | 0.04 |

2.10 Intermediate Precision:

Shenqi Fuzheng injections taken from the same batch were assayed according to the method described above, respectively, except for under the variable factors such as on different dates and by different analysts.

2.10.1 Different Analysis Dates:

Shenqi Fuzheng injections taken from the same batch were prepared according to the method of preparing test sample solution on different dates, respectively, and then three of the test sample solutions were injected in parallel, followed by extracting the characteristic peaks and marking the retention time. Peak 12 of astragaloside IV was taken as Peak S, and then the relative retention time of each of other characteristic peaks. The results showed that the RSD values of the relative retention time of each characteristic peak was all less than 1%, as shown in Table 6:

TABLE 6
Results of analysis on different dates

| | Date 1 | | | Date 2 | | | RSD (%) |
|---|---|---|---|---|---|---|---|
| 1 | 0.517 | 0.518 | 0.517 | 0.516 | 0.516 | 0.517 | 0.19 |
| 2 | 0.541 | 0.541 | 0.542 | 0.542 | 0.541 | 0.541 | 0.08 |
| 3 | 0.585 | 0.586 | 0.585 | 0.585 | 0.585 | 0.586 | 0.03 |
| 4 | 0.623 | 0.624 | 0.623 | 0.623 | 0.623 | 0.623 | 0.07 |
| 5 | 0.661 | 0.661 | 0.661 | 0.661 | 0.661 | 0.661 | 0.05 |
| 6 | 0.715 | 0.715 | 0.715 | 0.715 | 0.715 | 0.715 | 0.01 |
| 7 | 0.786 | 0.786 | 0.787 | 0.786 | 0.786 | 0.786 | 0.03 |
| 8 | 0.819 | 0.820 | 0.820 | 0.820 | 0.820 | 0.819 | 0.02 |
| 9 | 0.847 | 0.847 | 0.848 | 0.848 | 0.848 | 0.847 | 0.03 |
| 10 | 0.923 | 0.923 | 0.923 | 0.923 | 0.922 | 0.922 | 0.01 |
| 11 | 0.972 | 0.972 | 0.973 | 0.972 | 0.972 | 0.972 | 0.02 |
| S | 1.000 | 1.000 | 1.000 | 1.000 | 1.000 | 1.000 | 0.00 |
| 13 | 1.044 | 1.044 | 1.044 | 1.045 | 1.044 | 1.044 | 0.02 |
| 14 | 1.074 | 1.074 | 1.074 | 1.074 | 1.074 | 1.074 | 0.01 |
| 15 | 1.096 | 1.097 | 1.096 | 1.096 | 1.096 | 1.096 | 0.03 |
| 16 | 1.128 | 1.129 | 1.129 | 1.129 | 1.128 | 1.128 | 0.03 |
| 17 | 1.155 | 1.156 | 1.155 | 1.156 | 1.156 | 1.155 | 0.03 |
| 18 | 1.189 | 1.189 | 1.190 | 1.189 | 1.189 | 1.188 | 0.04 |

2.10.2 Different Analysts:

Shenqi Fuzheng injections taken from the same batch were prepared according to the method of preparing test sample solution by different analysts, respectively, and three of the test sample solution were injected in parallel, followed by extracting the characteristic peaks and marking the retention time. Peak 12 of astragaloside IV was taken as Peak S, and then the relative retention time of each of other characteristic peaks was calculated. The results showed that the RSD values of the relative retention time of each characteristic peak was all less than 1%, as shown in Table 7:

TABLE 7
Results of analysis by different analysts

| | Analyst 1 | | | Analyst 2 | | | RSD (%) |
|---|---|---|---|---|---|---|---|
| 1 | 0.516 | 0.518 | 0.519 | 0.519 | 0.519 | 0.519 | 0.18 |
| 2 | 0.542 | 0.544 | 0.544 | 0.544 | 0.543 | 0.544 | 0.16 |
| 3 | 0.586 | 0.588 | 0.588 | 0.589 | 0.586 | 0.587 | 0.18 |
| 4 | 0.623 | 0.625 | 0.625 | 0.626 | 0.624 | 0.625 | 0.16 |
| 5 | 0.661 | 0.663 | 0.663 | 0.663 | 0.661 | 0.663 | 0.17 |
| 6 | 0.714 | 0.716 | 0.717 | 0.717 | 0.714 | 0.716 | 0.19 |
| 7 | 0.786 | 0.788 | 0.789 | 0.789 | 0.785 | 0.788 | 0.19 |
| 8 | 0.819 | 0.821 | 0.822 | 0.822 | 0.819 | 0.821 | 0.15 |
| 9 | 0.848 | 0.848 | 0.849 | 0.849 | 0.847 | 0.848 | 0.07 |
| 10 | 0.922 | 0.922 | 0.923 | 0.923 | 0.922 | 0.923 | 0.03 |
| 11 | 0.972 | 0.972 | 0.972 | 0.972 | 0.972 | 0.972 | 0.03 |
| S | 1.000 | 1.000 | 1.000 | 1.000 | 1.000 | 1.000 | 0.00 |
| 13 | 1.044 | 1.044 | 1.044 | 1.044 | 1.044 | 1.044 | 0.02 |
| 14 | 1.074 | 1.074 | 1.074 | 1.074 | 1.074 | 1.074 | 0.01 |
| 15 | 1.096 | 1.095 | 1.095 | 1.095 | 1.096 | 1.096 | 0.02 |
| 16 | 1.128 | 1.129 | 1.129 | 1.128 | 1.128 | 1.128 | 0.02 |
| 17 | 1.155 | 1.155 | 1.155 | 1.155 | 1.155 | 1.156 | 0.03 |
| 18 | 1.189 | 1.188 | 1.188 | 1.188 | 1.189 | 1.189 | 0.04 |

### Example 2 Identification of Shenqi Fuzheng Injection

In recent years, as the use of Shenqi Fuzheng injection is increasing in clinical, some criminals are motivated by economic interest and counterfeit Shenqi Fuzheng injection with other varieties for sale to make huge profits, which results in a great negative impact on the brands for Shenqi Fuzheng injection, and has caused significant economic loss to these enterprises which produce and sell Shenqi Fuzheng injections legally. These counterfeit Shenqi Fuzheng injections have almost the same appearance as the real ones, so it is hard to distinguish the real from the fake.

In this example, the method described in Example 1 was adopted to test the certified Shenqi Fuzheng injection (provide by Livzon Group Limin Pharmaceutical Factory), a suspected sample and a Danshen injection, so as to establish the corresponding fingerprint profiles. The results were shown in FIG. 4. It can be seen that the fingerprint profile of the suspected sample is completely different from that of the certified Shenqi Fuzheng injection, the components of the counterfeit were inferred by using the precise molecular weight provided by mass spectrometry. It was substantially confirmed that the components of the counterfeit were derived from Danshen.

Thus, the Shenqi Fuzheng injection fingerprint profile established by ultra high pressure liquid chromatography-mass spectrometer (UHPLC-MS) can be used to identify the authenticity of a Shenqi Fuzheng injection in a fast and accurate manner, and can also be used to analyze the counterfeit qualitatively and substantially confirm the source. If there is any counterfeit Shenqi Fuzheng injection with DanShen injection by criminals, it can be identified according to the method described above, i.e. comparing the fingerprint profile of the injection with that of the certified Shenqi Fuzheng injection, inferring the components of the counterfeit by using the precise molecular weight provided by mass spectrometry, substantially confirming the component source of the counterfeit.

Therefore, application of the Shenqi Fuzheng injection UHPLC-MS fingerprint profile can avoid counterfeiting, and ensure normal production and good circulation of the Shenqi Fuzheng injection so as to protect legitimate rights and interests of manufactures.

### Example 3 Detection of Saponins in the Shenqi Fuzheng Injection by HPLC-ELSD Method

1. Preparation of Test Sample

In order to detect saponins in the test sample, the test sample was pretreated and concentrated. Therefore, Shenqi Fuzheng Injection (obtained from Livzon Group Limin Pharmaceutical Factory) was fractionated on a macroporous resin, eluted with water and different concentration of ethanol solution (firstly 30 v/v % ethanol solution and then 70 v/v % ethanol solution is used) to remove polysaccharides, protein and salts, and then concentrated, made up to the mark, filtered, 20 μl filtrate was injected into a liquid chromatography for analysis.

2. Preparation of Control Sample

(1) Selection of Control Sample

The main component of saponins in Shenqi Fuzheng Injection is astragaloside IV, therefore, in this study, astragaloside IV was selected as the control sample. Astragaloside IV was obtained from National Institutes for Food and Drug Control.

(2) Preparation of Control Solution Containing Astragaloside IV

An appropriate amount of astragaloside IV was accurately weighed, and then methanol was added to prepare a solution containing 0.10 mg/mL astragaloside IV as the control solution.

3. Detection

Detection was performed by high performance liquid chromatography (Chinese Pharmacopoeia, Part I, Appendix VI D, 2010 edition).

(1) Instruments and Reagents

Waters 2695 high performance liquid chromatograph, Waters 2420 ELSD detector, Empower2 workstation; Precisa 925M-202A electronic balance; chromatographically pure acetonitrile (TEDIA, USA); redistilled water.

(2) Chromatographic Conditions and Detection

\textcircled{1} Chromatographic Conditions

The chromatographic column used was Discovery C-18 (5 μm, 250×4.6 mm) column. Acetonitrile (A) and water (B) was used as mobile phases. Bivariate linear gradient elution was performed according to the table 16 below. Column temperature was 30° C. Flow rate was 1.0 ml/min; The temperature of drift tube was 55° C. Atomizing power was 60%. Gain value was 20. Gas pressure was 25 psig. The theoretical plate number should not be less than 3000 base on astragaloside IV.

TABLE 16

| Time (minute)) | Mobile phase A (%) | Mobile phase B (%) | Note |
|---|---|---|---|
| 0~30 | 12→25 | 88→75 | Equilibrating |
| 30~40 | 25→32 | 75→68 | the column |
| 40~55 | 32→55 | 68→45 | |
| 55~68 | 55→90 | 45→10 | |
| 68~72 | 90 | 10 | |
| 72~75 | 90→12 | 10→88 | |
| 75~83 | 12 | 88 | |

[symbol] Detection Method

20 μl of the solution of the control sample and the solution of the test sample were accurately taken respectively, and then injected into the high performance liquid chromatography to detect, the chromatogram for 70 minutes was recorded.

a. Determination of Mobile Phase

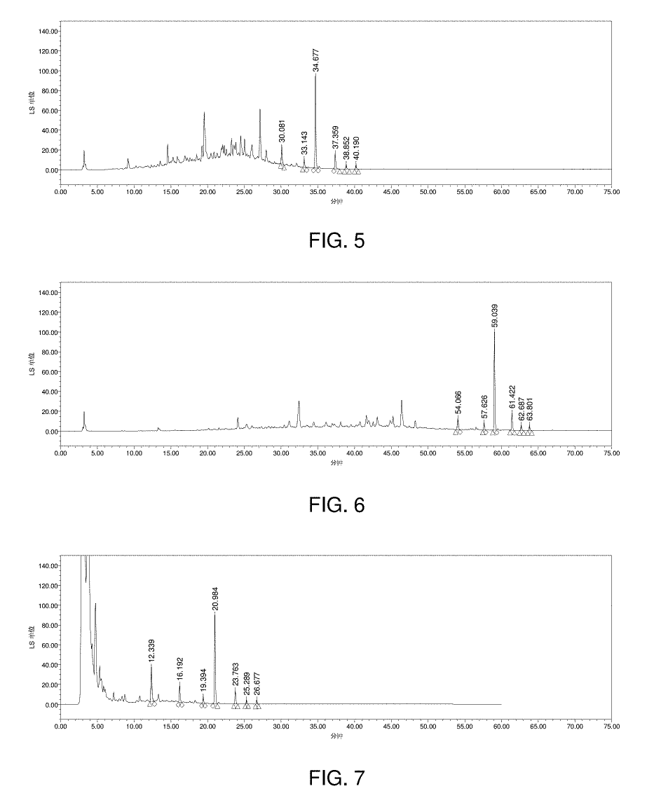

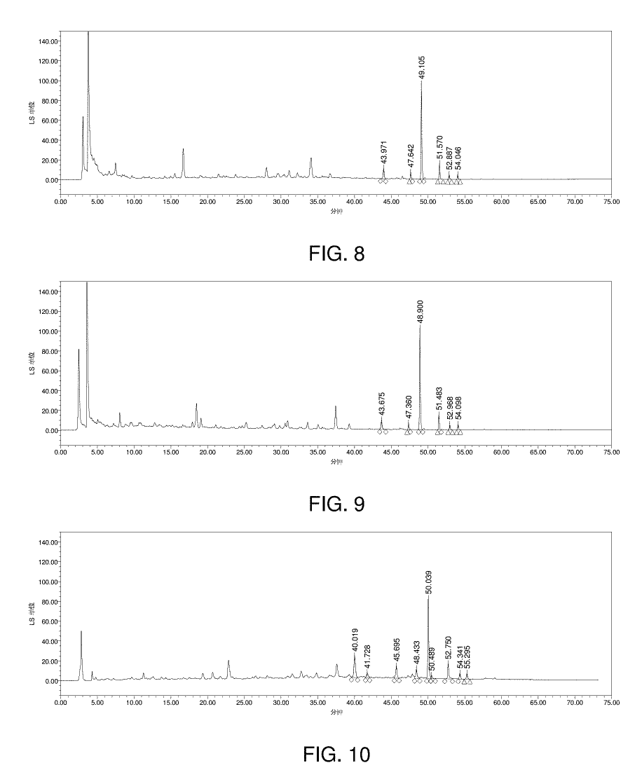

Acetonitrile and water were used as the mobile phase, the effect of different gradient elution programs (shown in Table 8-11) was evaluated and the results were shown in FIG. 5-8.

TABLE 8

| | A % | |
|---|---|---|
| Time (min) | (acetonitrile) | B % (water) |
| 0 | 2 | 98 |
| 80 | 100 | 0 |

TABLE 9

| | A % | |
|---|---|---|
| Time (min) | (acetonitrile) | B % (water) |
| 0 | 2 | 98 |
| 8 | 5 | 95 |
| 18 | 12 | 88 |
| 40 | 25 | 75 |
| 50 | 32 | 68 |
| 60 | 48 | 52 |
| 65 | 55 | 45 |
| 70 | 95 | 5 |
| 75 | 2 | 98 |

TABLE 10

| Time (min) | A % (acetonitrile) | B % (water) |
|---|---|---|
| 0 | 25 | 75 |
| 5 | 25 | 75 |
| 30 | 55 | 45 |
| 35 | 55 | 45 |
| 40 | 90 | 10 |
| 45 | 90 | 10 |
| 50 | 25 | 75 |
| 60 | 25 | 75 |

TABLE 11

| | A % | |
|---|---|---|
| Time (min) | (acetonitrile) | B % (water) |
| 0 | 12 | 88 |
| 30 | 25 | 75 |
| 40 | 32 | 68 |
| 55 | 55 | 45 |
| 68 | 90 | 10 |
| 72 | 90 | 10 |

It can be seen from the results that the condition shown in table 11 is desirable for good peak shape and flat baseline.

b. Determination of Chromatographic Column

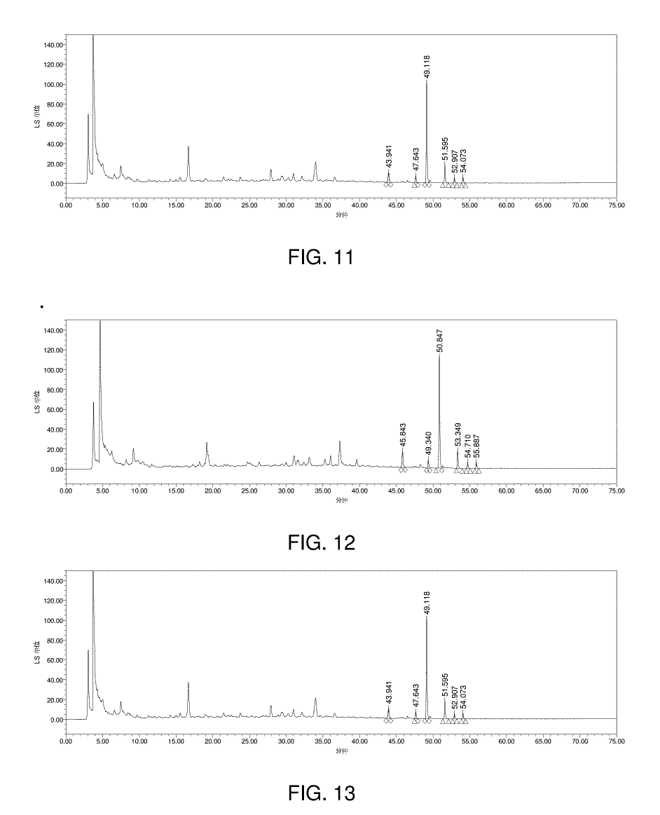

The effect of different types of column (Kromasil column, Diamonsil column and Discovery column) were evaluated was evaluated respectively. The results were shown in FIG. 9-11. It can be seen from the results that Discovery column was superior to other columns in separating degree and peak shape.

c. Determination of Flow Rate

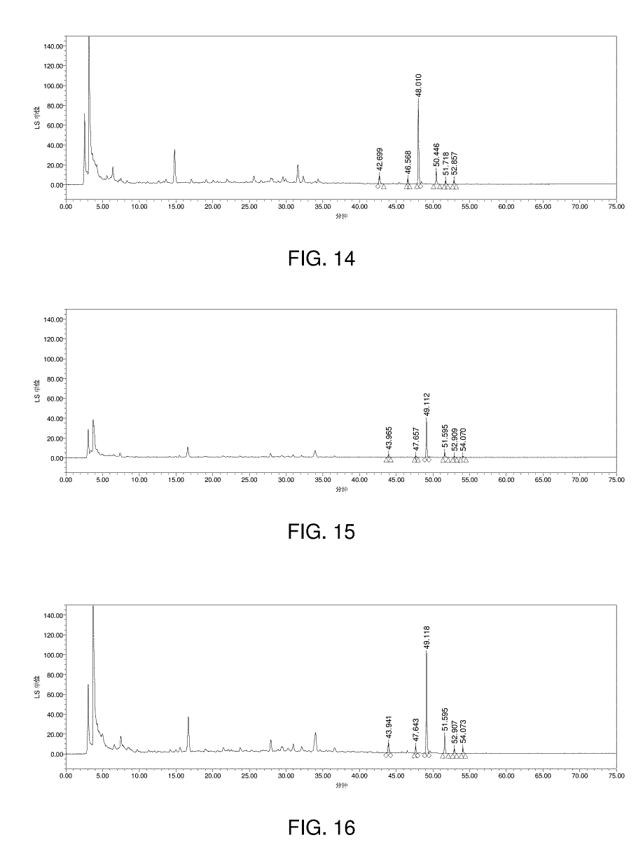

The effect of different flow rate (0.8, 1.0 and 1.2 ml/min) was evaluated respectively. The results were shown in FIG. 12-14. It can be seen from the results that the separating degree and peak number were desirable under the flow rate of 1.0 ml/min.

d. Determination of Injection Volume

The effect of different injection volume (10 μl and 20 μl) was evaluated. The results were shown in FIG. 15-16. It can be seen from the results that 20 μl of injection volume can meet the requirement.

e. Determination of Pretreatment Method

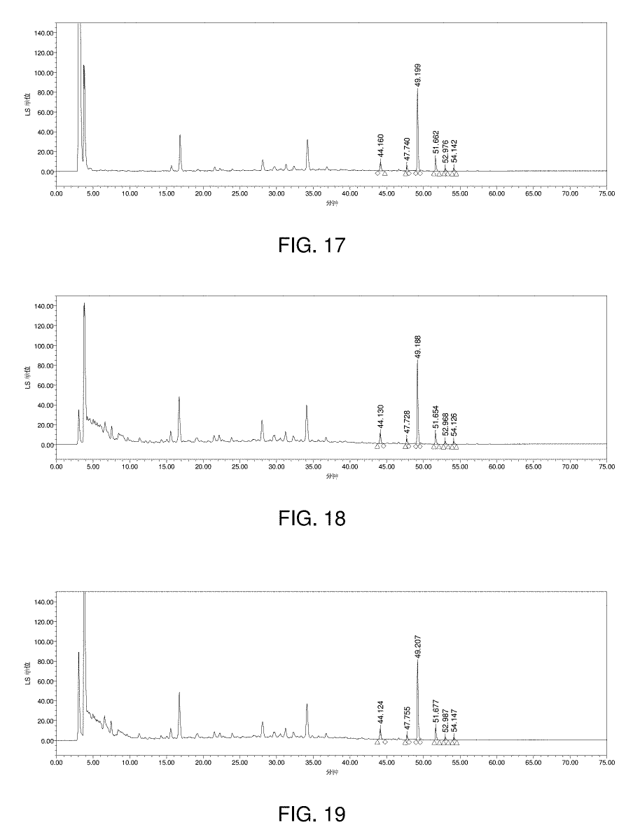

The effect of different pretreatment method (extraction with n-butanol, purification on D₁₀₁ macroporous resin directly and purification on D₁₀₁ macroporous resin after concentration) was evaluated. The results were shown in FIG. 17-19. It can be seen from the results that the chromatogram obtained by purification on D₁₀₁ macroporous resin directly was better, and the method was simple and easy to operate.

f. Determination of Collecting Time

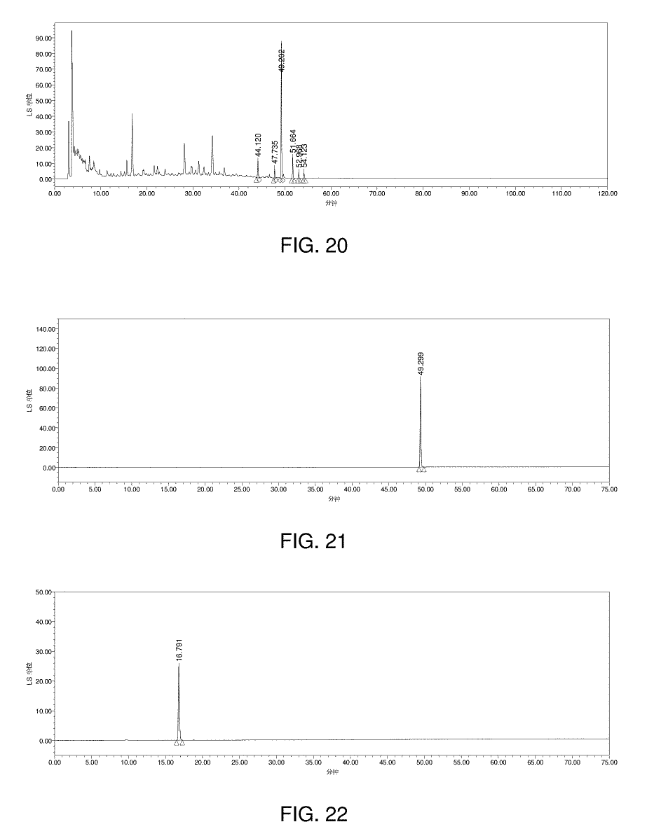

20 μl of the test sample solution was accurately taken, and then injected into the high performance liquid chromatography to detect, the chromatogram for 120 minutes was recorded (see FIG. 20). It can be seen from the results that there were no chromatographic peaks after 70 minutes.

g. Chromatogram of Control Samples

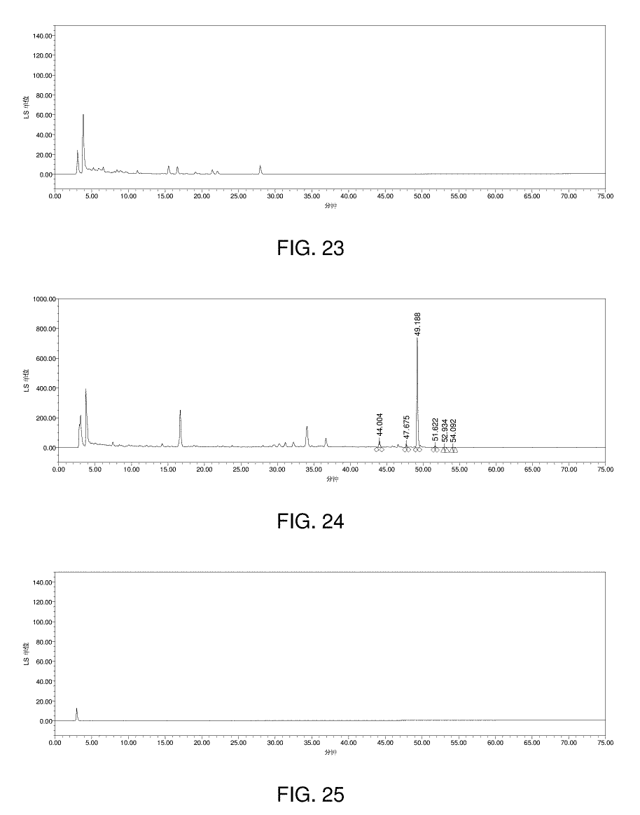

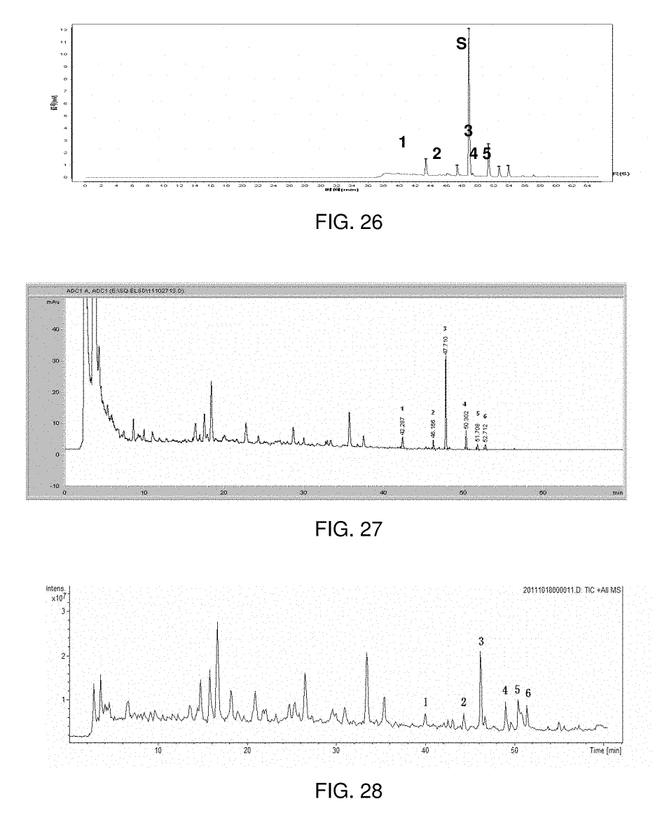

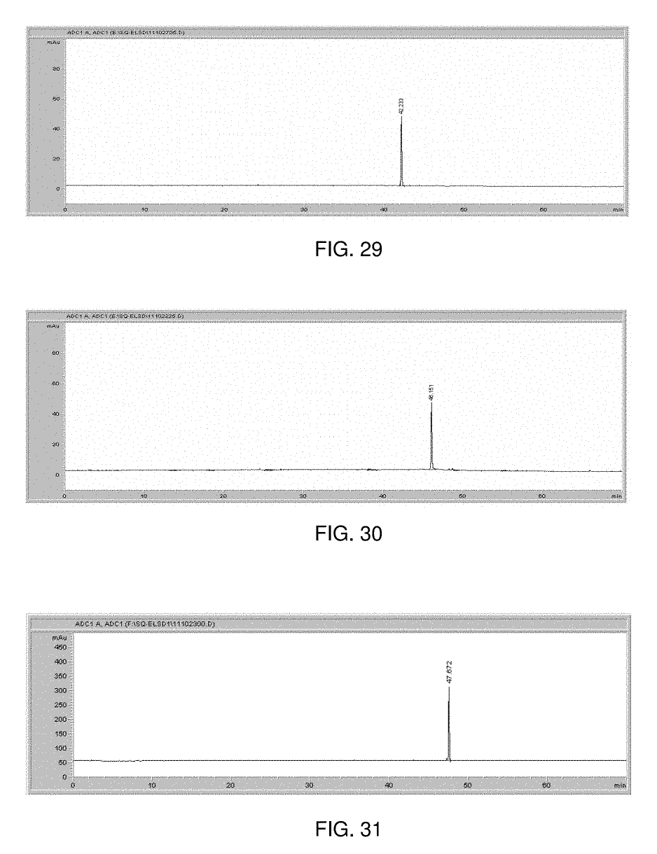

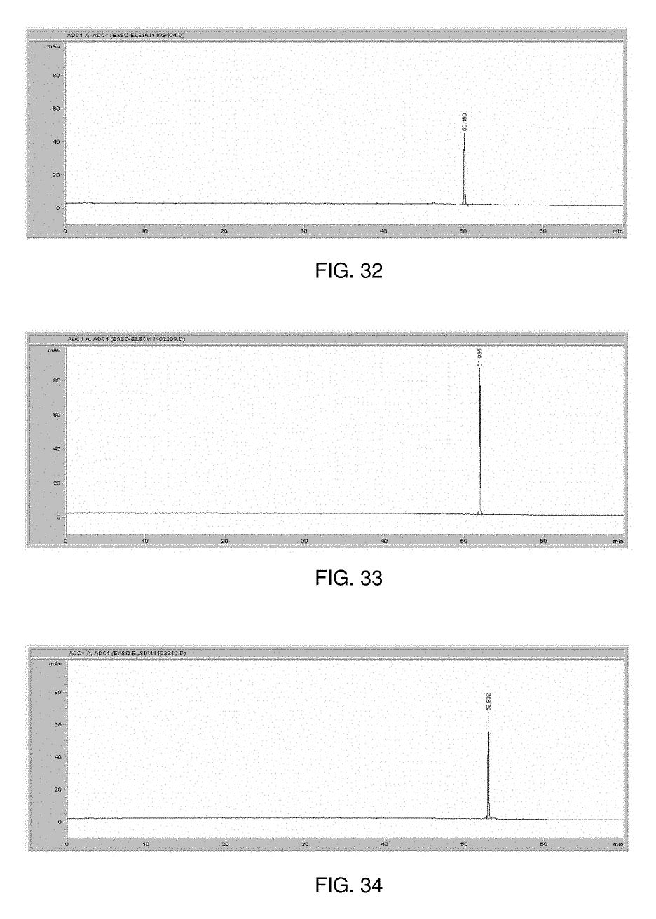

The chromatogram of control samples were shown in FIG. 21-25.

h. Methodological Investigation

Precision assay: The test sample solution from the same batch of Shenqi Fuzheng injection (1310907) was prepared according to the method of preparing the test sample solution, injected for continuously 6 times (20 μl each time), the chromatogram of each time was evaluated. The results were shown in table 12. The results showed that the RSD values of the peak area of each characteristic peak were all less than 3%, which meet the requirements of fingerprint.

TABLE 12

| Peak | | | | | | | |
|---|---|---|---|---|---|---|---|
| Nos. | Sample 1 | Sample 2 | Sample 3 | Sample 4 | Sample 5 | Sample 6 | RSD (%) |
| 1 | 106726 | 107456 | 105375 | 105299 | 106357 | 103779 | 1.23 |
| 2 | 47169 | 48259 | 48067 | 46995 | 49608 | 48083 | 1.94 |
| 3 | 676756 | 678533 | 697947 | 695351 | 680992 | 681028 | 1.33 |
| 4 | 111327 | 114908 | 112780 | 115608 | 110807 | 114194 | 1.72 |
| 5 | 32065 | 31124 | 32754 | 31971 | 31653 | 31742 | 1.69 |
| 6 | 32526 | 33830 | 34814 | 34093 | 34722 | 33257 | 2.59 |

Stability Assay:

This experiment is used to evaluate the Stability of the test samples. The test sample solution from the same batch of Shenqi Fuzheng injection (1310907) was prepared according to the method of preparing the test sample solution, the detection were performed at 0 hour, 4 hour, 12 hours, 16 hours and 24 hours respectively. The results were shown in table 13. The results showed that the RSD values of the peak area of each characteristic peak were all less than 3%, which meet the requirements of fingerprint.

TABLE 13

| Peak Nos. | 0 h | 4 h | 12 h | 16 h | 24 h | RSD (%) |
|---|---|---|---|---|---|---|
| 1 | 103110 | 103915 | 104753 | 104582 | 100935 | 1.50 |
| 2 | 47487 | 46044 | 45048 | 47213 | 45963 | 2.15 |
| 3 | 674093 | 682926 | 674569 | 678924 | 694969 | 1.25 |
| 4 | 105154 | 108038 | 105012 | 103491 | 108602 | 2.05 |
| 5 | 31502 | 31883 | 31634 | 30861 | 31443 | 1.20 |
| 6 | 32204 | 32363 | 32467 | 31785 | 33315 | 1.73 |

Repeatability Assay

Six Shenqi Fuzheng injection of the same batch were taken (1310907), prepared according to the method of preparing the test sample solution, and then injected, respectively, the respective chromatogram was recorded. The results were shown in table 14. The results showed that the RSD values of the peak area of each characteristic peak were all less than 3%, which meet the requirements of fingerprint.

TABLE 14

| Peak | | | | | | | |
|---|---|---|---|---|---|---|---|
| No. | Sample 1 | Sample 2 | Sample 3 | Sample 4 | Sample 5 | Sample 6 | RSD (%) |
| 1 | 103915 | 105463 | 105785 | 102326 | 101069 | 106726 | 2.10 |
| 2 | 46044 | 48468 | 47416 | 46653 | 45984 | 47169 | 2.00 |
| 3 | 682926 | 688582 | 668801 | 675287 | 679817 | 676756 | 1.00 |
| 4 | 108038 | 108716 | 111045 | 107912 | 108836 | 111327 | 1.37 |
| 5 | 31883 | 31708 | 31019 | 31492 | 30739 | 32065 | 1.63 |
| 6 | 32363 | 32557 | 33979 | 32916 | 33906 | 32526 | 2.18 |

4. Fingerprint Spectrum and Technical Parameters

Similarity

Six chromatographic peaks of 1, 2, S, 3, 4 and 5 in the control sample and test sample were corrected, and then the similarity was calculated, which should not be less than 0.95.

Setting of Parameters for Similarity

(1) Software: “Similarity Evaluation System for Chromatographic Fingerprint of Traditional Chinese Medicine (Version 2004A)” compiled by the National Pharmacopoeia Society was used.

(2) Reference spectrum: Referring to document of “Reference Spectrum for Calculation. Scp”.

(3) Time window width: 0.10

(4) Data cutting: the chromatographic peaks before 38 minutes and after 65 minutes in the fingerprint spectrum were cut.

(5) Correction of chromatographic peaks: Multi-point correction between the reference spectrum and six peaks of 1, 2, S, 3, 4 and 5 was performed.

Identification of main chromatographic peaks Six main saponins in Shenqi Fuzheng Injection were identified by HPLC-ELSD and HPLC-MSⁿ.

5. Determination of Limit Value of Similarity

The fingerprints of 15 batches of Shenqi Fuzheng injections were investigated. The results of calculation for similarity were shown in Table 15. Considering the actual condition and the statistical result of table 15 and other many factors, the limit value of similarity was provisionally set as 0.85.

TABLE 15

| Batch Nos. | Similarity |
|---|---|
| 121201 | 1.000 |
| 121202 | 0.996 |
| 121203 | 0.998 |
| 130101 | 0.999 |
| 130102 | 0.999 |
| 130803 | 0.999 |
| 130804 | 0.993 |
| 130805 | 0.990 |
| 1305443 | 0.998 |
| 1310025 | 0.999 |
| 1310026 | 0.998 |
| 1310027 | 0.997 |
| 1310907 | 0.995 |
| 1310908 | 0.995 |
| 1310909 | 0.996 |

Minimum: 0.990
Maximum: 1.000
Average: 0.997

The method provided by the present invention for establishing Shenqi Fuzheng injection fingerprint profile was described in details hereinbefore. The principles and embodiments of the present invention are described herein with reference to some specific examples, however, the examples described above are only intended to help understand the method and the core idea of the present invention. It should be noted that, for those skilled in the art, a number of improvements and modifications can be introduced to the present invention, without departing from the principles of the present invention, and these improvements and modifications shall fall into the scope defined by the appended claims.

What is claimed is:

1. A system for identifying Shenqi Fuzheng injection, the system comprising:
means for establishing a profile of a sample to be tested;
means for establishing a characteristic fingerprint profile of Shenqi Fuzheng injection as a standard fingerprint profile; and
means for comparing the profile of the sample to be tested with the standard fingerprint profile to distinguish between authentic Shenqi Fuzheng injection and counterfeit Shenqi Fuzheng injection;
wherein, the standard fingerprint profile comprises 18 characteristic peaks, and a retention time and a mass number of the 18 characteristic peaks are as follows:
Peak 1:7.1 min (471.2083), Peak 2: 7.5 min (491.1195), Peak 3: 8.1 min (441.1919), Peak 4: 8.6 min (309.1555), Peak 5: 9.2 min (187.0976), Peak 6: 9.9 min (441.1766), Peak 7: 10.9 min (593.1876), Peak 8: 11.3 min (507.1508), Peak 9: 11.7 min (463.1610), Peak 10: 12.7 min (991.5119), Peak 11: 13.4 min (991.5119), Peak 12: 13.7 min (829.4591), Peak 13: 14.4 min (871.4697), Peak 14: 14.8 min (871.4697), Peak 15: 15.1 min (871.4697), Peak 16: 15.5 min (913.4650), Peak 17: 15.9 min (913.4650), and Peak 18: 16.3 min (913.4650);
wherein Peak 2 and Peak 12 are calycosin glucoside and astragaloside IV, respectively;
wherein the Shenqi Fuzheng injection characteristic fingerprint profile takes a control astragaloside IV as a reference peak, by which a relative retention time of each characteristic peak is calculated, as follows:
Peak 1: 0.52, Peak 2: 0.54, Peak 3: 0.59, Peak 4: 0.62, Peak 5: 0.66, Peak 6: 0.72, Peak 7: 0.79, Peak 8: 0.82, Peak 9: 0.85, Peak 10: 0.92, Peak 11: 0.97, Peak 12: 1.00, Peak 13: 1.04, Peak 14: 1.07, Peak 15: 1.10, Peak 16: 1.13, Peak 17: 1.16, and Peak 18: 1.19;
wherein, when comparing, the sample is identified as authentic Shenqi Fuzheng injection if the relative retention time fluctuates within ±5% of the above specified values, and a ratio of an area of calycosin glucoside peak and the astragaloside IV peak to an area of a corresponding reference peak is 0.5-1.5.

2. The system of claim 1, wherein the means for establishing the profile of the samples to be tested and the means for establishing the characteristic fingerprint profile of Shenqi Fuzheng injection as the standard fingerprint profile each independently comprise ultra-high pressure liquid chromatography-mass spectrometer.

3. The system of claim 1, further comprising means for detecting saponins in Shenqi Fuzheng injection.

4. A method for identifying Shenqi Fuzheng injection, comprising the following steps:
step (1): establishing a profile of the samples to be tested;
step (2): establishing a characteristic fingerprint profile of Shenqi Fuzheng injection as the standard fingerprint profile;
step (3): comparing the profile of the samples to be tested in step (1) with the standard fingerprint profile in step (2) to distinguish between true and false;
wherein, the standard fingerprint profile in step (2) comprises 18 characteristic peaks, and the retention time and the mass number of the 18 characteristic peaks are as follows:
Peak 1:7.1 min (471.2083), Peak 2: 7.5 min (491.1195), Peak 3: 8.1 min (441.1919), Peak 4: 8.6 min (309.1555), Peak 5: 9.2 min (187.0976), Peak 6: 9.9 min (441.1766), Peak 7: 10.9 min (593.1876), Peak 8: 11.3 min (507.1508), Peak 9: 11.7 min (463.1610), Peak 10: 12.7 min (991.5119), Peak 11: 13.4 min (991.5119), Peak 12: 13.7 min (829.4591), Peak 13: 14.4 min (871.4697), Peak 14: 14.8 min (871.4697), Peak 15: 15.1 min (871.4697), Peak 16: 15.5 min (913.4650), Peak 17: 15.9 min (913.4650), Peak 18: 16.3 min (913.4650);
wherein Peak 2 and Peak 12 are calycosin glucoside and astragaloside IV, respectively;
wherein the Shenqi Fuzheng injection characteristic fingerprint profile takes the control astragaloside IV as a reference peak, by which the relative retention time of each characteristic peak is calculated, as follows:
Peak 1: 0.52, Peak 2: 0.54, Peak 3: 0.59, Peak 4: 0.62, Peak 5: 0.66, Peak 6: 0.72, Peak 7: 0.79, Peak 8: 0.82, Peak 9: 0.85, Peak 10: 0.92, Peak 11: 0.97, Peak 12: 1.00, Peak 13: 1.04, Peak 14: 1.07, Peak 15: 1.10, Peak 16: 1.13, Peak 17: 1.16, Peak 18: 1.19;
wherein, when comparing, the relative retention time fluctuates within ±5% of the above specified values, and the ratio of the area of the calycosin glucoside peak and the astragaloside IV peak to the area of the corresponding reference peak is 0.5-1.5.

5. The method according to claim 4, wherein, the profile of the samples to be tested in step (1) was established by the following method, which comprising testing the samples to be tested by ultra-high pressure liquid chromatography-mass spectrometer, wherein the method comprises the following steps:
(1) preparation of control solution: accurately weighing an appropriate amount of calycosin glucoside or astragaloside IV, and then adding methanol to prepare a solution containing 0.004 mg of calycosin glucoside per ml or 0.006 mg of astragaloside IV per ml, respectively;
(2) preparation of test sample solution: filtering the samples to be tested through a 0.22 μm microporous filter membrane;
(3) determination: accurately aspirating 5 μl of the control solution or the test sample solution, respectively, and then injecting the solutions into a ultra-high pressure liquid chromatography-mass spectrometer, conducting determination according to the following conditions to obtain the fingerprint profile;
wherein the chromatographic conditions include the followings:
chromatographic column: Agilent Zorbax Eclipse Plus C18, 2.1 mm×100 mm, 1.8 μm;
mobile phase: mobile phase A is 0.1% (v/v) formic acid aqueous solution, mobile phase B is 0.1% (v/v) formic acid acetonitrile solution;
using gradient elution according to the following elution program, wherein the proportions of the mobile phases are all volume percentages:
0-0.5 min, mobile phase A is 95%, mobile phase B is 5%;
0.5-10 min, mobile phase A is 95%-75%, mobile phase B is 5%-25%;
10-15 min, mobile phase A is 75%-45%, mobile phase B is 25%-55%;
15-18 min, mobile phase A is 45%-0%, mobile phase B is 55%-100%;
18-20 min, mobile phase A is 0%, mobile phase B is 100%.
flow rate: 0.35 ml/min;
column temperature: 40° C.;
the ion source is an ESI source, and detection is operated in negative ion mode;
atomized gas pressure: 35 psig;
dry gas temperature: 350° C.;
dry gas flow rate: 10 L/min;
capillary voltage: 3,500 V;
voltage at capillary exit: 135 V.

6. The method according to claim 4, wherein, the Shenqi Fuzheng injection characteristic fingerprint profile in step (2) was established by the following method, which comprises testing Shenqi Fuzheng injection by ultra-high pressure liquid chromatography-mass spectrometer, wherein the method comprises the following steps:
(1) preparation of control solution: accurately weighing an appropriate amount of calycosin glucoside or astragaloside IV, and then adding methanol to prepare a solution containing 0.004 mg of calycosin glucoside per ml or 0.006 mg of astragaloside IV per ml, respectively;
(2) preparation of test sample solutions: filtering Shenqi Fuzheng injection through a 0.22 μm microporous filter membrane;
(3) determination: accurately aspirating 5 μl of the control solution or the test sample solution, respectively, and then injecting the solutions into a ultra-high pressure liquid chromatography-mass spectrometer, conducting determination according to the following conditions to obtain the Shenqi Fuzheng injection fingerprint profile;
wherein the chromatographic conditions include the followings:
chromatographic column: Agilent Zorbax Eclipse Plus C18, 2.1 mm×100 mm, 1.8 μm;
mobile phase: mobile phase A is 0.1% (v/v) formic acid aqueous solution, mobile phase B is 0.1% (v/v) formic acid acetonitrile solution;
using gradient elution according to the following elution program, wherein the proportions of the mobile phases are all volume percentages:
0-0.5 min, mobile phase A is 95%, mobile phase B is 5%;
0.5-10 min, mobile phase A is 95%-75%, mobile phase B is 5%-25%;
10-15 min, mobile phase A is 75%-45%, mobile phase B is 25%-55%;
15-18 min, mobile phase A is 45%-0%, mobile phase B is 55%-100%;
18-20 min, mobile phase A is 0%, mobile phase B is 100%.
flow rate: 0.35 ml/min;
column temperature: 40° C.;
the ion source is an ESI source, and detection is operated in negative ion mode;
atomized gas pressure: 35 psig;
dry gas temperature: 350° C.;
dry gas flow rate: 10 L/min;
capillary voltage: 3,500 V;
voltage at capillary exit: 135 V;
(4) determination of common characteristic peaks to obtain the standard fingerprint profile: comparing multiple Shenqi Fuzheng injection fingerprint profiles obtained from the above method, picking out 18 common characteristic peaks as the standard fingerprint profile.

7. A method for detecting saponins in Shenqi Fuzheng injection, the method comprising:
preparing chromatographic conditions for HPLC-ELSD as followings:
chromatographic column: Discovery C-18, 5 μm, 250×4.6 mm;
mobile phase: mobile phase A is acetonitrile, mobile phase B is water; and
using gradient elution according to the following elution program, wherein the proportions of the mobile phases are all volume percentages:
0-30 min, mobile phase A is 12-25%, mobile phase B is 88-75%;
30-40 min, mobile phase A is 25%-32%, mobile phase B is 75%-68%;
40-55 min, mobile phase A is 32%-55%, mobile phase B is 68%-45%;
55-68 min, mobile phase A is 55%-90%, mobile phase B is 45%-10%;
68-72 min, mobile phase A is 90%, mobile phase B is 10%.

8. The method according to claim 7, wherein, the chromatographic conditions also include the followings:
flow rate: 1.0 ml/min;
column temperature: 30° C.;
injection volume: 20 μl.

9. The method according to claim 7, wherein the conditions of the ELSD detector include the followings:
the temperature of drift tube was 55° C.;
atomizing power was 60%;
gain value was 20;
gas pressure was 25 psig.

10. The method according to claim 7, further comprising preparation of control solutions by the following steps:
accurately weighing an appropriate amount of astragaloside IV, and adding methanol to prepare a solution containing 0.10 mg of astragaloside IV per ml.

11. The method according to claim 7, further comprising preparation of a test sample solution by the following step:
fractioning the Shenqi Fuzheng Injection on a macroporous resin,
eluting the Shenqi Fuzheng Injection with water and, firstly 30 v/v % ethanol solution and then 70 v/v % ethanol solution to remove polysaccharides, protein and salts,
concentrating the Shenqi Fuzheng Injection,
setting the volume, and
filtering the Shenqi Fuzheng Injection.
